(19) United States
(12) Patent Application Publication
(10) Pub. No.: US 2007/0198026 A1
(43) Pub. Date: Aug. 23, 2007

(54) Medical device support and stabilizer

(76) Inventor: Joseph C. Cauthen, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/360,920

(22) Filed: Feb. 23, 2006

(51) Int. Cl. A61F 11/00

(52) U.S. Cl. 606/108

(57) ABSTRACT

A device for stabilizing hydrocephalus shunt valves, or other medical apparatus, to reduce or eliminate repositioning, particularly when positioned in the fascia or subcutaneous tissues within a body. The shunt valve stabilizer of the subject invention is particularly useful for stabilizing lumboperitoneal (LP) shunt valves, but could be used with other types of shunt valves or medical devices susceptible to undesired movement within or on a body.

### BACKGROUND OF INVENTION

Hydrocephalus is an abnormal condition caused by failure of absorption and subsequent accumulation of cerebrospinal fluid (CSF) within the ventricles of the brain and spine. As the CSF builds up it causes the ventricles to enlarge and the pressure inside the head and spine to increase. When there is a CSF production-absorption imbalance, the resulting pressure increase can cause loss of oxygen content within brain tissue and loss of brain function, specifically memory loss, dementia, unstable walking patterns, and loss of urinary control.

There are two classifications of hydrocephalus. These two classifications are referred to as communicating and noncommunicating. Communicating hydrocephalus is caused by inadequate absorption of CSF due to an inability of the CSF to pass through the arachnoidal villi for subsequent transport into the blood stream. The circulation pathways are competent from the ventricles inside of the brain to the fluid spaces just below the fourth ventricle, but become blocked in the arachnoidal villi causing CSF to collect within the ventricles. The excessive accumulation of CSF results in an abnormal enlargement of the brain ventricles and can lead to brain damage.

Noncommunicating hydrocephalus typically refers to hydrocephalus that develops as a result of a blockage in the normal circulation of cerebrospinal fluid (CSF) within the brain. In most cases it refers to a blockage between the third and fourth ventricles, referred to as an “aqueductal obstruction”, usually at the level of the Aqueduct of Sylvius. This can be due to scarring of this passage (e.g., “aqueductal stenosis”), a tumor (e.g., “tectal glioma”), or other factors.

The most effective treatment for communicating hydrocephalus is the implantation of a shunt system having a shunt, a programmable or pressure differential valve, an entry catheter, and an exit catheter for drainage. These shunt systems are usually designed to drain excess cerebrospinal fluid from the ventricles of the brain to either the peritoneal cavity or the right atrium of the heart. Shunt systems are usually also designed to function using the pressure differential between the brain and another part of the body and to regulate the drainage flow using a programmable valve.

The most common shunt system uses a ventriculoperitoneal (VP) shunt. With the VP shunt, a surgical procedure provides an opening in the skull through which an entry catheter is introduced and passed through the brain tissue into the ventricles. The catheter is attached to a pump or valve, which is positioned subcutaneously against the exterior of the skull, to control fluid flow. A second exit catheter, usually also subcutaneous in position, is also attached to the pump and leads to the peritoneal cavity.

An alternative procedure, which avoids this invasive cranial procedure, is to use a lumboperitoneal (LP) shunt. With the LP shunt system, CSF is drained from the lumbar region of the spine to the peritoneal cavity. While the LP shunt system eliminates cranial invasion, it is considered an unconventional and less reliable procedure because of the tendency for the programmable valve of the LP shunts to rock, twist, tear, or otherwise move out of position. Most LP shunts are positioned subcutaneously against the side of the body, essentially perpendicular to the plane of the spine. Movement of the LP shunt from the optimal subcutaneous position can make monitoring and adjusting of the programmable shunt settings difficult or impossible. Ultimately, further surgical procedures may become necessary to reposition and reattached the LP shunt and/or valve.

There exists a need for a means to stabilize the valves on hydrocephalus shunt systems, and similar devices that require precise positioning within or on a body. In particular, the ability to stabilize lumboperitoneal (LP) shunt valves can increase the efficacy of this procedure and reduce the need to utilize invasive cranial procedures.

### BRIEF SUMMARY

The subject invention addresses the need to reduce or prevent undesirable repositioning of hydrocephalus shunt valves. Specifically, the subject invention provides a device that is capable of stabilizing a variety of styles and types of shunt valves, or similar devices, to reduce or eliminate undesirable repositioning of shunts or valves, particularly when positioned in the fascia or subcutaneous tissues within a body.

The shunt valve stabilizer of the subject invention is particularly useful for stabilizing lumboperitoneal (LP) shunt valves, but can be used with other types of shunt valves, or other medical devices, susceptible to undesired movement within or on a body.

In a preferred embodiment, the present invention provides a subcutaneous platform and housing designed to receive the valve of a shunt system, or other functional mechanism, and maintain the valve in a desired position once secured to the fascia or subcutaneous tissues. The device is capable of maintaining a position underlying and against the subcutaneous tissues.

In general, the device comprises an essentially flattened platform with a sheath-like housing for containing a shunt valve. The device and the shunt valve therein can be secured to the underlying fascia or other subcutaneous tissues utilizing surgical sutures. The dimensions of the device are such that, once sutured into position, it lends stability to a shunt valve by resisting movement, particularly rolling or twisting movements that move or hide the face of the valve so that it can no longer be monitored or adjusted when necessary.

### BRIEF DESCRIPTION OF DRAWINGS

FIG. 1 is a top view of an embodiment of the medical device stabilizer of the subject invention.

FIG. 2 is a front view of the embodiment of the medical device stabilizer shown in FIG. 1.

FIG. 3 is a side view of an embodiment of the medical device stabilizer shown in FIG. 1.

FIG. 4 is a top view of the embodiment shown in FIG. 1 further illustrating the use, for example, of medical sutures or staples to secure the medical device stabilizer.

FIG. 5 is a top view of another embodiment of the medical device stabilizer of the subject invention, further comprising a skirting.

FIG. 6 is a front view of the embodiment of the medical device stabilizer shown in FIG. 5.

FIG. 7 is a side view of the embodiment of the medical device stabilizer shown in FIG. 5.

FIG. 8 is a top view of a further embodiment of the medical device stabilizer of the subject invention comprising a tubing slit in the housing cover.

FIG. 9 is top view of a further embodiment of the medical device stabilizer of the subject invention comprising a tubing slit contiguous with an adjustment window in the housing cover.

FIG. 10 is front view of the embodiment of the medical device stabilizer shown in FIG. 9.

FIG. 11 is a side view the embodiment of the medical device stabilizer shown in FIG. 9.

FIG. 12 is a bottom view of an embodiment of the medical device stabilizer of the subject invention, further comprising a skirting material attached to the periphery of the device.

FIG. 13 is a bottom view of an alternative embodiment of the medical device stabilizer of the subject invention, wherein the device is affixed to a swatch of skirting material.

FIG. 14 a top view of an embodiment of the medical device stabilizer of the subject invention illustrating one type of shunt valve system that can be utilized with the subject medical device stabilizer.

FIG. 15 a top view of an embodiment of the medical device stabilizer of the subject invention illustrating an alternative shunt valve system that can be utilized with the subject medical device stabilizer.

FIG. 16 is a top view of an embodiment of the medical device stabilizer of the subject invention illustrating a covering within the adjustment window.

FIG. 17 is a top view of an embodiment of the medical device stabilizer of the subject invention illustrating the use of, for example, surgical sutures and/or staples for securing the medical device stabilizer, as well as securing a shunt valve to the stabilizer.

### DETAILED DISCLOSURE

The subject invention provides new and advantageous devices that stabilize a variety of styles and types of shunt valves (or similar devices) to reduce or eliminate undesirable repositioning of shunts or valves, particularly when positioned in the fascia or subcutaneous tissues within a body.

The shunt valve stabilizer of the subject invention is particularly useful for stabilizing lumboperitoneal (LP) shunt valves, but can be used with other types of shunt valves, or other medical devices, susceptible to undesired movement within or on a body.

In a preferred embodiment, the present invention provides a subcutaneous platform and housing designed to receive the valve of a shunt system and maintain the valve in a desired position once secured to the fascia or subcutaneous tissues. The device maintains a position underlying and against the subcutaneous tissues.

The device typically comprises a flat platform with a sheath-like housing for containing a shunt valve. The device and the shunt valve therein can be secured to the underlying fascia or other subcutaneous tissues utilizing surgical sutures. The dimensions of the device are such that, once sutured into position, it lends stability to a shunt valve by resisting movement, particularly rolling or twisting movements that move or hide the face of the valve so that it can no longer be monitored or adjusted when necessary.

With reference to the attached figures, which illustrate certain embodiments of the subject invention, it can be seen that the shunt valve stabilizer comprises a housing fixedly attached to a relatively flexible material for supporting and/or connecting the housing to the surrounding fascia and/or subcutaneous tissues. In practice, a shunt valve device can be inserted into the housing and secured to the housing with surgical sutures or similar means. The support apparatus can then be affixed to surrounding fascia or other subcutaneous tissues with additional surgical sutures, staples, or similar means.

It can be seen from the embodiments shown in the Figures that the housing 2 is a sheath-like covering comprising a housing cover 4 fixedly connected to a housing base 8. The circumferential shape of the housing 2 may comprise any of a variety of shapes depending upon the particular area of the body to which it will be secured, the type of devices to be used with the housing 2, etc. However, in a preferred embodiment, the housing circumference has sufficiently curved, arcuate or otherwise smooth lines to reduce or prevent damage to surrounding tissues.

The housing 2 is designed as a generic receptacle capable of receiving a variety of styles, sizes and shapes of shunt valves. Therefore, the circumferential shape can be a in any of variety of shapes, including squared, rounded, ovate, or any other polygonal shape. In a preferred, embodiment, the circumferential shape of the stabilizer device 10 is a generic form capable of containing and supporting a variety of shunt valve styles and types, or other similar medical devices, to reduce or eliminate undesired movement.

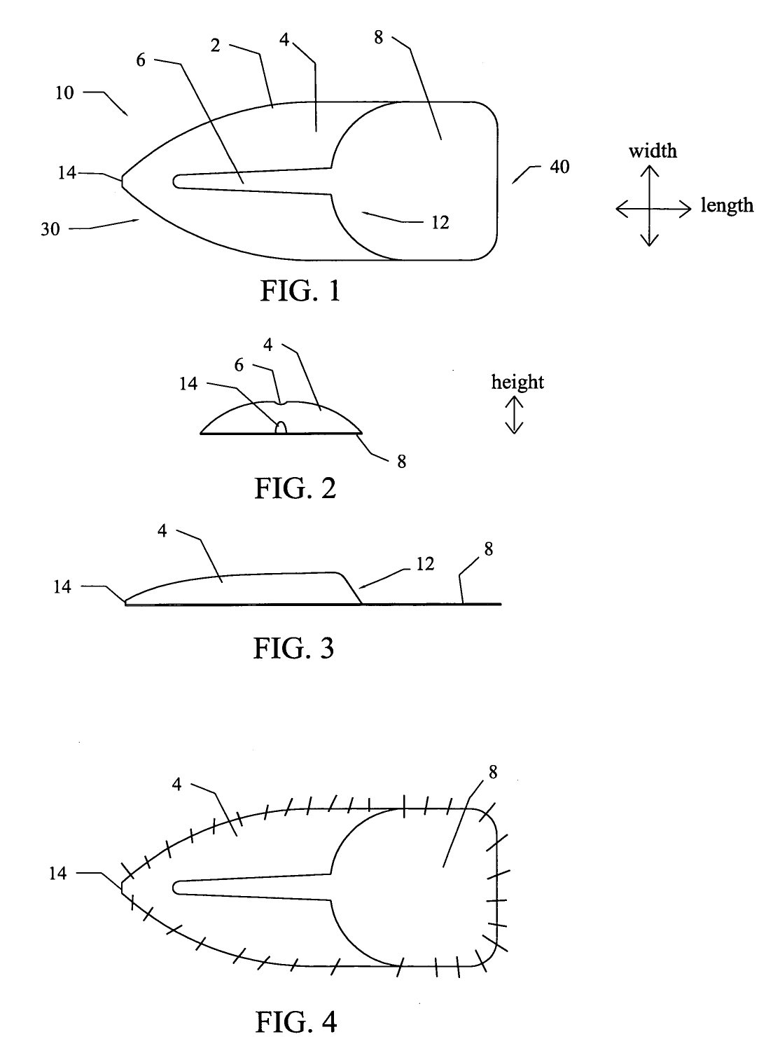

In a preferred embodiment, the front end 30 of the stabilizer device 10 is narrowed and widens in a tapering fashion towards the back end 40 of the stabilizer device 10, for example as shown in FIGS. 1, 5, and 12. This shape allows the housing 2 to accept and support a large variety of shapes and sizes of shunt valves. However, it would be well within the skill of a person trained in the art to create a stabilizer device with alternative circumferential shapes based upon the description provided herewith.

As mentioned above, the housing 2 comprises a housing cover 4 fixedly attached to a housing base 8. The housing base 8 is essentially a platform capable of resting against or beside underlying subcutaneous tissues. In an alternative embodiment, the stabilizer device 10 is utilized outside a body, such that the housing base 8 can rest against, and, if desired, be affixed to, an area outside a body, for example against the skin, to support a shunt valve or other similar device. To assist in the comfort, placement and stability of the housing 2, the underside of the housing base 8, which would be in contact with a body, may further have a longitudinal and/or latitudinal concavity to assist in fitting against body tissues. In a preferred embodiment, the housing base 8 is generally flattened, but comprises a semi-flexible material capable of sufficient conformation to the shape of the underlying subcutaneous or other tissues, but also sufficiently resistant to over flexing so as to avoid susceptibility to rolling, twisting or other undesired movement of the stabilizer device 10.

The housing base 8 may comprise any of a variety of biocompatible materials. Thus, the base may comprise, for example, various types of silicones, plastics, fabrics, meshes, polymeric fabrics or meshes, combinations thereof, etc.

The dimensions of the housing base 8 can vary depending upon the anticipated shunt valves or other similar devices expected to be used with the stabilizer device 10. However, it may be preferable for the entire functional length of a shunt valve or similar device used with the stabilizer device 10 to able to be positioned on the housing base 8. As will be discussed below, shunt valves or similar devices utilized with the stabilizer device will be secured, at least partially, to the housing base 8. Further, as mentioned previously, it is preferable for the stabilizer device 10 to be as generic as possible to accommodate the large variety of shunt valves and other devices expected to be used with the subject invention. Therefore, in one embodiment, the length of the housing base 8, from the front end 30 to the back end 40 is approximately 5 centimeters to about 10 centimeters, and the width is approximately 1.5 centimeters to about 5 centimeters. In a preferred embodiment, the length of the housing base 8, from the front end 30 to the back end 40 is approximately 6 centimeters to about 8 centimeters, and the width is approximately 2 centimeters to about 4 centimeters. In a more preferred embodiment, the length of the housing base 8, from the front end 30 to the back end 40 is approximately 7 centimeters, and the width approximately 3 centimeters.

Extending along a portion of the housing base 8 is a housing cover 4, for example as shown in FIGS. 1 and 3. The housing cover 4 may be fixedly attached to the housing base 8 by techniques that are well-known to those with skill in the art. The housing cover 4 is essentially a sheath-like cover over a portion of the housing base 8 that forms a housing cavity 12 capable of providing support to a shunt valve or similar device positioned therein. The housing cover extends across, at least a portion of, the length of the base. In a preferred embodiment, the housing cover 4 extends from approximately 50% to 100% of the length of the housing base 8. However, in a more preferred embodiment, the housing cover 4 extends from approximately 65% to about 85% of the length of the housing base 8. In an even more preferred embodiment, the housing cover 4 extends from approximately 45% to about 75% of the length of the housing base 8.

Shunt valves, and many other medical devices very often have one or more tubes, wires, or other extensions. For example, lumboperitoneal (LP) and ventriculoperitoneal (VP) shunts have tubing extending from either or both ends of the shunt valve for carrying fluids away from the brain and into, most often, the peritoneal cavity. Other devices may also have various tubes, wires, or similar extensions. Therefore, the stabilizer device 10 should be capable of supporting shunt valves, or similar devices, without interfering with, blocking or crimping the tubing, wires, etc. extending therefrom. Thus, in one embodiment, the housing further comprises one or more tubing apertures 14 through which one or more tubes can extend. In a preferred embodiment, the one or more tubing apertures 14 are positioned at or near the front end 30 of the housing 2, for example as shown in FIGS. 2 and 6. The size and shape of the tubing aperture 14 will depend upon the dimensions of the items to be inserted therein. In one embodiment, the tubing aperture is semi-circular with a radius of approximately 0.5 centimeter to about 1 centimeter.

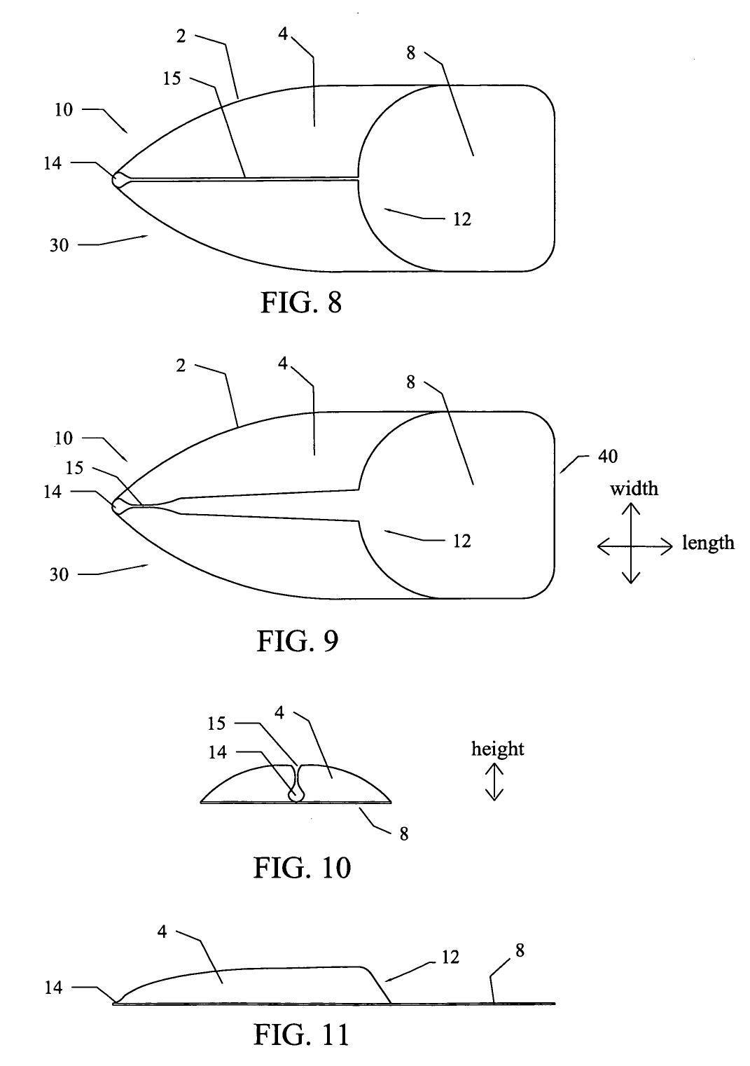

In an alternative embodiment, the one or more tubing apertures 14 can be contiguous with one or more tubing slits 15 that extend along the length of the housing cover 4. In a preferred embodiment, the one or more tubing apertures 14 can be contiguous with one tubing slit 15 that extends along the length of the housing cover, for example as shown in FIG. 8. In this embodiment, tubing affixed to a medical device can be pushed or pressed through the tubing slit 15, which negates the need to disassemble or disconnect tubing already affixed to a medical device. In a further embodiment, the housing cover 4, or that portion thereof around the tubing slit 15, comprises a sufficiently flexible material to accommodate pressing or pushing medical tubing through the tubing slit 15 without damaging said tubing. In a still further embodiment, the housing cover 4 can have sufficient flexibility so that one or the other side can be pressed or pushed downwards as tubing is inserted therethrough, but return to its original position, essentially parallel to the opposite side, to prevent tubing from exiting back through the tubing slit 15. Alternatively, the tubing slit 15 can be sufficiently wide enough so that the flexibility of tubing or other material allows it to be pressed or pushed through the width of the tubing slit 15. It would be well within the skill of a person trained in the art to create alternative tubing apertures 14 or tubing slits 15 in or on the housing 2 to accommodate a variety of medical devices that could be used with the device of the subject invention. By way of example, the tubing aperture 14 can be designed as a channel or tube within or extending from the housing cover 4, through which the tubing of a medical device could be threaded or positioned through. Further, one or more tubing apertures 14 can be positioned in different areas of the housing to further accommodate a variety of medical devices. Such alternatives are contemplated to be within the scope of the present invention.

The height of the housing cover 4 in relation to the housing base 8 may vary, again, depending upon the type of shunt valve or similar device expected to be utilized therewith. But, in a preferred embodiment, the height of the housing cover 4 is approximately 0.4 centimeters to about 0.8 centimeters above the level of the housing base 8. However, in a more preferred embodiment, the height of the housing cover 4 is approximately 0.5 centimeters to about 0.7 centimeters above the level of the housing base 8. In a most preferred embodiment, the height of the housing cover 4 is approximately 0.6 centimeters above the level of the housing base 8.

The housing cover 4 may comprise any of a variety of, preferably biocompatible, materials, which can be, for example, various types of silicones, plastics, fabrics, meshes, polymeric fabrics or meshes, combinations or composites thereof, etc. In a preferred embodiment, the housing cover 4 comprises a biocompatible material capable of being fixedly connected to the housing base 8. In one embodiment, the housing cover 4 and the housing base 8 comprise the same material. A person with skill in the art can determine appropriate materials for the composition of the housing base 8 and the housing cover 4, which may include one or more or a combination of materials. In a preferred embodiment, the housing cover 4 comprises a semi-rigid material.

The device of the subject invention is particularly suitable for use with lumboperitoneal (LP) and/or ventriculoperitoneal (VP) shunts. These shunts are often placed subcutaneously under the scalp (VP shunts) in a parallel orientation to the spine, or along the side of the body, below the ribcage in a nearly perpendicular orientation to the spine. These shunts also require periodic adjustment that is accomplished in a variety of ways, but usually, and preferably, without actually contacting the shunt valve. Often, the shunt valves are magnetically adjustable, which allows adjustment through skin, fascia, etc. However, to make adjustments, the control valve mechanism must be facing outward from the body at nearly a perpendicular orientation to the external programming device. The device of the subject invention is useful for maintaining the orientation of such devices. But, depending upon the materials of the stabilizing device 10, the magnetic, or other, forces used to make adjustments may be inhibited or blocked.

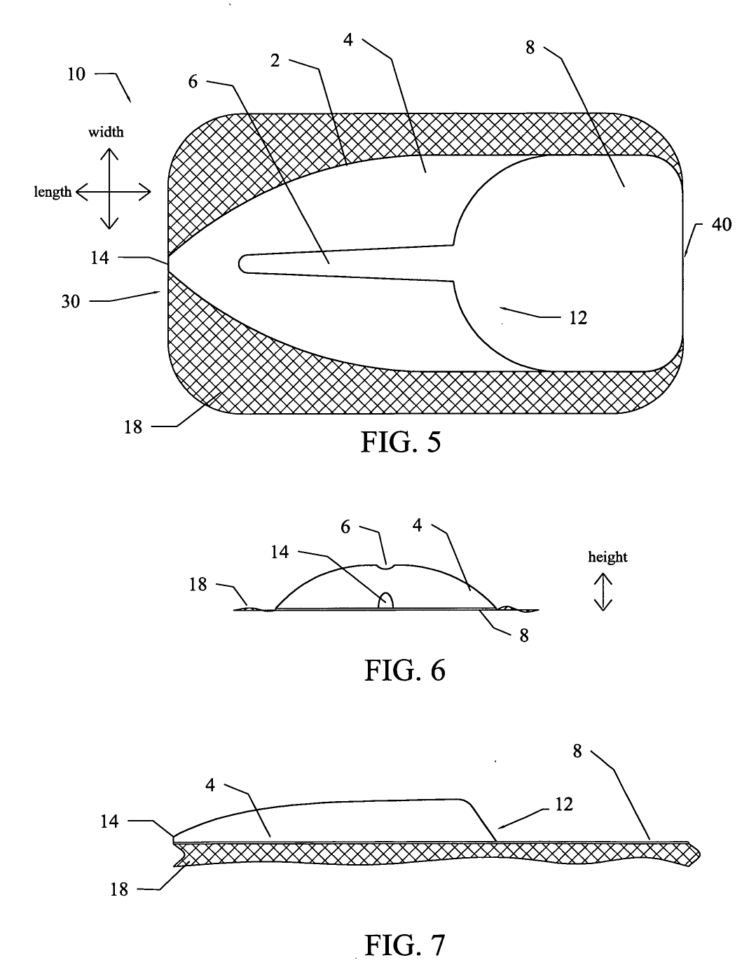

Thus, the stabilizer device 10 may further comprise an opening or adjustment window 6 that allows electromagnetic access to an adjustable valve mechanism. The adjustment window 6 is essentially an opening through the housing cover 4. The size of the adjustment window 6 may be altered to accommodate the dimensions utilized for the housing 2 and/or the housing cover 4. The size of the adjustment window 6 may also depend upon the size and/or type of shunt valve or other device used in the stabilizer device 10, the type of adjustments required and the device used to make the adjustments. In one embodiment, the adjustment window 6 is an opening within the housing, usually near the apex of the housing cover 4. However, in a preferred embodiment, the adjustment window 6 is a slit or channel within the housing cover 4 at or near the apex that extends from the edge of the opening of the housing cavity 12 to approximately ½ to about ⅔ the length of the housing cover 4 and is approximately 0.2 centimeter to about 0.5 centimeter at its widest point, for example as shown in FIGS. 1 and 5.

In this embodiment, the tubing slit 15 discussed above can be contiguous with the adjustment window 6, for example as shown in FIG. 9. Thus, the tubing aperture 14 can be contiguous with the tubing slit 15, which can be contiguous with the adjustment window 6. In use, a medical device with tubing in place can still be inserted into the stabilizer device 10 without disassembling or disconnecting any of the tubing from the medical device.

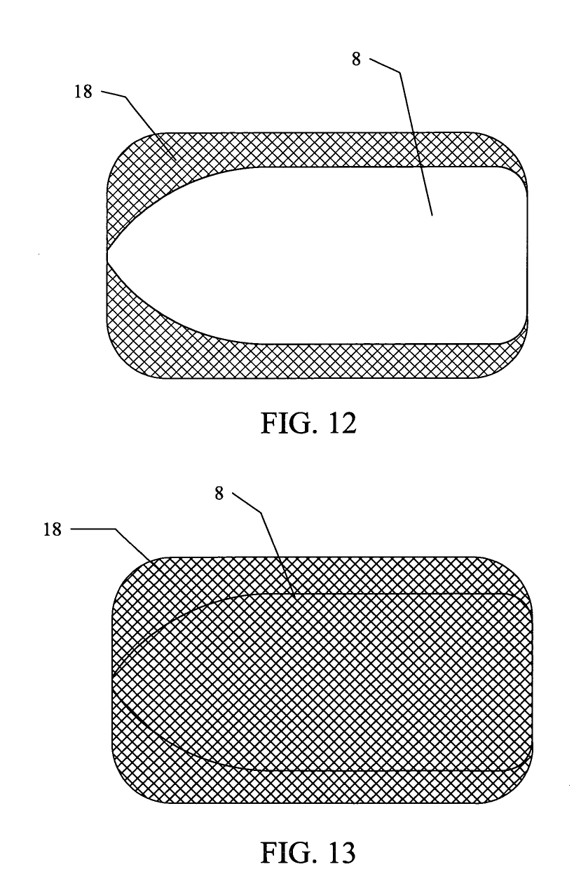

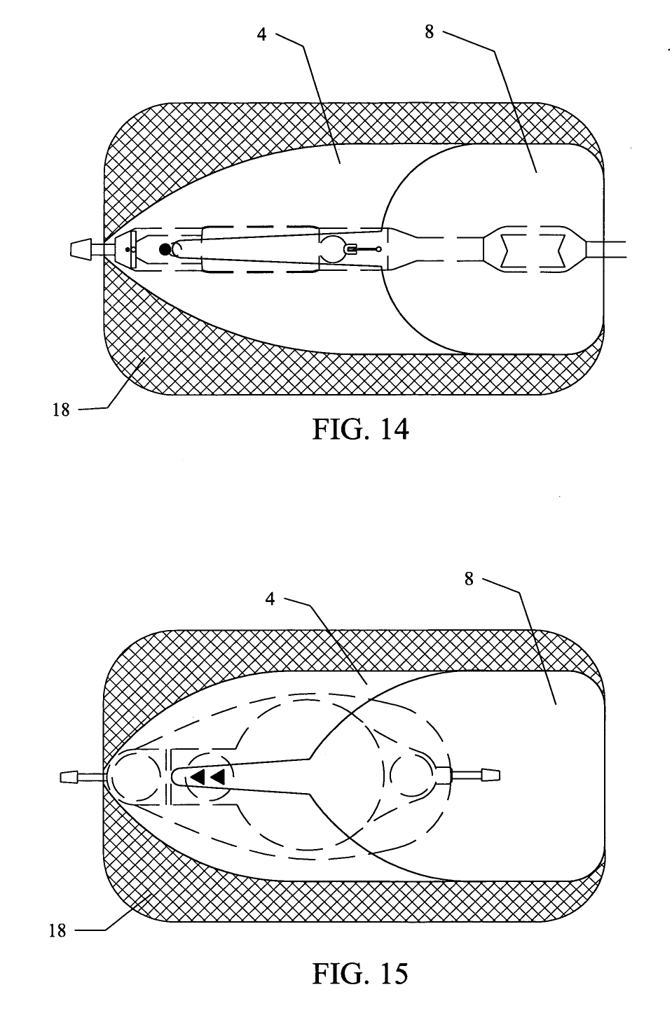

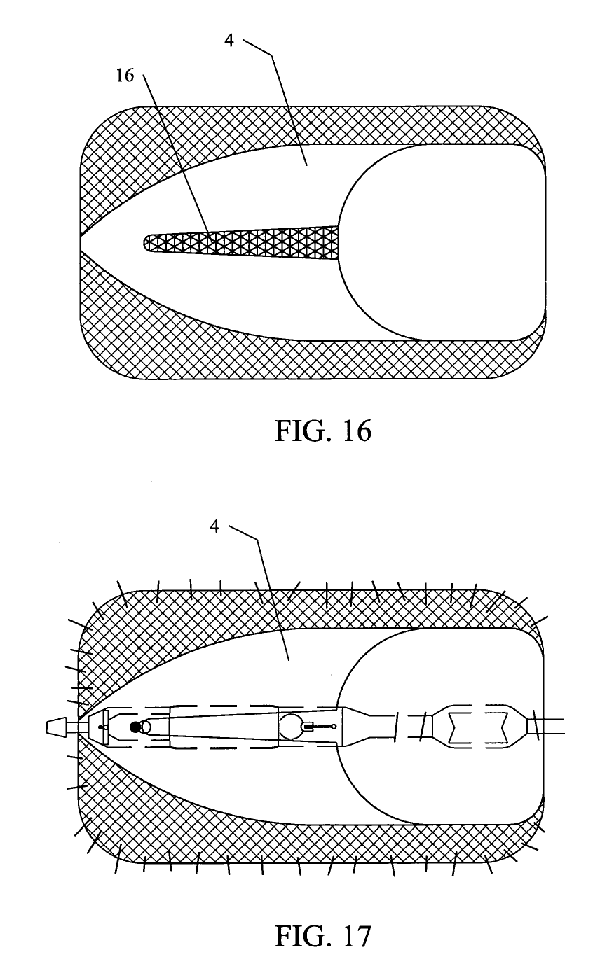

In an alternative embodiment, the adjustment window 6 further comprises a covering 16, for example a mesh-like material, within or covering the adjustment window 6, for example as shown in FIG. 12. Such a covering 16 could lend support the sides of the housing cover 4, but would not hinder to block electromagnetic access to any valve adjustment mechanism or similar device within the housing 2. Such a covering may also protect the face of the adjustment mechanism. However, this embodiment would not allow for the insertion of a medical device with tubing already connected, as discussed above. A wide variety of other sizes, shapes and coverings useful for an adjustment window 6 in the subject invention will be apparent to those skilled in the art after reviewing the description herein.

The stabilizer device 10 of the subject invention can be utilized within or on a body. However, the stabilizer device 10 described herein is particularly suited for use within a body, particularly within the subcutaneous tissues. In a preferred embodiment, the stabilizer device 10 is secured to the subcutaneous tissues and fascia. In a further preferred embodiment, a shunt valve mechanism or other device is further secured to the stabilizer device 10. Securing the stabilizer device 10 and any mechanisms therein reduces or eliminates the possibility of these devices being twisted, turned, rotated or otherwise moved away from the optimal position. FIGS. 10 and 11 illustrate examples of different types of shunt valves that can be utilized with the stabilizer device 10 of the subject invention.

The most common way to secure the stabilizer device 10 is with the use of multiple surgical sutures or staples around the periphery of the device. However, for securing a shunt valve or other device to the stabilizer device 10, it may be preferably to use surgical sutures or other fixation means. In this embodiment, surgical sutures or staples are used to penetrate the housing cover 4 and/or the housing base 8, depending upon the location of the sutures or staples.

A variety of options exist that can facilitate the use of sutures with the device of the subject invention. For example, the stabilizer device 10 could be manufactured from a material that allows for penetration of surgical needles. Thus, in one embodiment, the device could simply be sewn to surrounding tissues by securing the periphery with surgical sutures. In an alternative embodiment, openings could be provided around the periphery of the stabilizer device 10 for passing through a surgical needle and sutures. In an even further alternative embodiment, various types of loops or eyelets could be provided around the periphery of the device which would also allow for passing through a surgical needle and sutures. Similar means could be provided for securing a shunt valve or other device to the stabilizer device 10.

However, in a preferred embodiment, the circumference of the stabilizer device 10, or one or more sections thereof, is surrounded by a fabric or mesh-like material skirting 18, for example Marlex Mesh™. Such material skirting 18 can be affixed to one or more sections of, or the entire circumference of, the stabilizer device 10, for example as shown in FIG. 8. Alternatively, the underside of the housing base 8 can be fixedly attached to a swatch of the fabric-like material to provide a skirting 18 around the periphery, for example as shown in FIG. 9. A person with skill in the art will be able to readily determine the most effective means for affixing such skirting 18 to the stabilizer device 10, as well as the proper peripheral length and/or dimensions depending upon placement. To secure a shunt valve or other device to the stabilizer device 10, sutures can be passed around the shunt valve and through the housing base 8. If the housing base 8 comprises a material that is not conducive to surgical needle penetration, sutures could be passed across the width of the housing base 8 and secured through the skirting 18 around the periphery of the housing 2.

The advantage of utilizing a skirting 18 of suitable fabric or mesh-like material 18, like Marlex Mesh™, is that the stabilizer device can be more easily sewn to the tissues utilizing as many, or as few, sutures or staples as necessary and in the best positions for the area of placement. For example, in some situations it may be preferable to install sutures in a perpendicular and/or parallel position relative to the stabilizing device 10. It may also be preferable to install sutures in a variable fashion, for example, extra sutures may be installed at the top of the device and fewer sutures at the bottom. By utilizing a skirting 18 around the periphery, a medical professional will have a wider range of choices as to where and how to place and secure the stabilizer device of the subject invention. FIG. 13 illustrates how the device may be secured using surgical sutures or staples with this embodiment.

It should be understood that the examples and embodiments described herein are for illustrative purposes only and that various modifications or changes in light thereof will be suggested to persons skilled in the art and are to be included within the spirit and purview of this application.

1. A method for securing a medical apparatus within a patient's body utilizing a sterile device comprising one or more biocompatible materials, wherein said device further comprises:
a housing base;
a housing cover fixedly connected to the housing base; and
a skirting fixedly connected to at least a portion of the housing base and extending beyond the periphery of the housing base,
wherein said method further comprises positioning the medical apparatus within said device and securing said device to a tissue within the body thereby securing the medical apparatus.

2. The method, according to claim 1, wherein the device is secured within the patient's body with sutures and/or medical staples.

3. The method, according to claim 2, wherein the sutures and/or medical staples are connected to the skirting to secure the device within a body.

4. The method, according to claim 1, wherein the housing cover of the device encompasses the length of the housing base.

5. The method, according to claim 1, wherein the housing cover of the device encompasses approximately 45% to about 85% of the housing base.

6. The method, according to claim 1, wherein said device comprises a biocompatible material that permits magnetic or electromagnetic access to a medical apparatus therein.

7. The method, according to claim 1, wherein said device comprises a housing base approximately 5 cm to about 10 cm in length and approximately 1.5 cm to about 5 cm in width.

8. The method, according to claim 1, wherein said device comprises a housing cover with a height of approximately 0.4 cm to about 0.8 cm above the housing base.

9. The method, according to claim 1, wherein the device further comprises a housing cover having an adjustment window.

10. The method, according to claim 9, wherein said adjustment window further comprises a covering, wherein said covering permits magnetic or electromagnetic access to a medical apparatus therein.

11. The method, according to claim 1, wherein said device further comprises at least one tubing aperture.

12. The method, according to claim 11, further comprising at least one tubing slit contiguous with the at least one tubing aperture.

13. The method, according to claim 11, wherein said at least one tubing aperture is located at or near that end of the housing cover opposite the opening to the housing cavity, wherein said tubing aperture is capable of accommodating tubing, wires or other extensions which may be present on a medical apparatus positioned therein.

14. The method, according to claim 1, wherein said skirting of the device comprises a biocompatible polymeric material.

15. The method, according to claim 1, wherein said skirting of the device comprises a biocompatible mesh material.

16. The method, according to claim 1, wherein said skirting of the device comprises Marlex™ mesh.

17. The method, according to claim 1, wherein said medical device is a hydrocephalus shunt valve.

18. The method, according to claim 1, wherein said medical device is a lumboperitoneal hydrocephalus shunt valve.

19. A sterile device made from biocompatible materials wherein said device comprises:
a housing base;
a housing cover fixedly connected to the housing base; and
a skirting fixedly connected to at least a portion of the housing base and extending beyond the periphery of the housing base,
such that said device can be secured to tissues within a body to stabilize a medical apparatus positioned therein.

20. The device, according to claim 19, wherein the housing cover encompasses the length of the housing base.

21. The device, according to claim 19, wherein the housing cover encompasses approximately 45% to about 85% of the housing base.

22. The device, according to claim 19, wherein said biocompatible material permits magnetic or electromagnetic access to a medical apparatus therein.

23. The device, according to claim 19, wherein the housing base is approximately 5 cm to about 10 cm in length and approximately 1.5 cm to about 5 cm in width.

24. The device, according to claim 19, wherein the height of the housing cover is approximately 0.4 cm to about 0.8 cm above the housing base.

25. The device, according to claim 19, wherein the housing cover further comprises an adjustment window.

26. The device, according to claim 25, wherein said adjustment window further comprises a covering, wherein said covering permits magnetic or electromagnetic access to a medical apparatus therein.

27. The device, according to claim 19, further comprising at least one tubing aperture.

28. The device, according to claim 27, further comprising at least one tubing slit contiguous with said at least one tubing aperture.

29. The device, according to claim 27, wherein said at least one tubing aperture is located at or near the end of the housing cover opposite the opening to the housing cavity, wherein said tubing aperture is capable of accommodating tubing, wires or other extensions which may be present on a medical apparatus positioned therein.

30. A sterile device of one or more biocompatible materials comprising:
a housing base; and
a housing cover fixedly connected to the housing base;
wherein said one or more biocompatible materials permit the device to be secured to tissues within a body utilizing one or more standard surgical techniques in order to stabilize a medical apparatus positioned therein.

31. The device, according to claim 30, wherein a standard surgical technique utilizes suture and/or medical staples.
